(19) United States
(12) Patent Application Publication
(10) Pub. No.: US 2005/0158728 A1
(43) Pub. Date: Jul. 21, 2005

(54) Laser device and method for collapsing hybridization substrate

(75) Inventors: Julio P. Focaracci, San Mateo, CA (US); Douwe D. Haga, Redwood City, CA (US); Nelson G. Todd, San Mateo, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 10/762,991

(22) Filed: Jan. 21, 2004

(51) Int. Cl. C12Q 1/68; C12M 1/34; H01L 21/00

(52) U.S. Cl. 435/6; 435/287.2; 438/1

(57) ABSTRACT

The present application relates to an apparatus and method for laser collapsing of a porous material on a nonporous substrate.

### FIELD

The present application relates to an apparatus and method for preparing a nonporous substrate with a porous layer for hybridization of an array.

### INTRODUCTION

Hybridization of various biological samples can utilize different types of test arrays. Test arrays for hybridization can include a grid of test sites within a bounded reaction well. Various capture reagents can flood the test array to support a reaction between various capture reagents in a hybridization fluid and various specific binding components. However, the flow of the hybridization fluid should be confined to a specific isolated area of a porous layer coupled to a nonporous substrate. This area can be bounded around a subset of individual test sites and/or around the entire test array. To create a boundary, porous material is typically removed around the test sites and/or test array. Typical removal methods contact the porous layer to effect removal of portions of the porous layer. This removal creates a boundary of the array and/or test sites that can act to restrict the flow of hybridization fluid. However, contact with the porous layer creates inefficiencies such as unpredictable transfer of residue from the nonporous layer to the removal device, thereby producing inconsistent results and introducing additional steps of frequent cleaning. It is desirable to treat a portion of the porous layer without contacting the porous layer.

All patents, applications, and publications mentioned here and throughout the application are incorporated in their entireties by reference herein and form a part of the present application.

### SUMMARY

According to various embodiments, the present teachings can provide a method for preparing a substrate for hybridization, the method including positioning a porous layer on the substrate, and collapsing a moat in the porous layer with a laser, wherein the moat is adapted to bound a portion of the porous layer on which an array can be positioned.

According to various embodiments, the present teachings can provide a method for manufacturing, including providing a substrate including a porous layer, wherein the porous layer is adapted for depositing an array, providing a laser assembly, wherein the laser assembly includes laser, and collapsing the moat in the porous layer with the laser.

According to various embodiments, the present teachings can provide a method for preparing a hybridization chamber, including providing a substrate including a porous layer with a moat collapsed with a laser, positioning an array on a portion of the porous layer bound by the moat, and positioning a gasket in the moat to provide a nonporous seal.

According to various embodiments, the present teachings can provide an apparatus for preparing a hybridization substrate, including a laser assembly adapted to collapse a moat in a porous layer on the substrate, and a galvanometer scan assembly adapted to position laser light from the laser assembly on the porous layer.

According to various embodiments, the present teachings can provide a laser assembly, including a laser adapted to collapse a moat in a porous layer of a hybridization substrate, a mechanism to position the laser light on a portion of the porous layer.

According to various embodiments, the present teachings can provide a substrate for hybridization, including a porous layer, wherein the porous layer is adapted for depositing an array, and a moat in the porous layer, wherein the moat is collapsed by laser.

According to various embodiments, the present teachings can provide an apparatus for preparing a substrate for hybridization including means for providing a moat in a porous layer on a substrate by laser means, wherein the porous layer is adapted for depositing an array.

According to various embodiments, the present teachings can provide a system for automated preparation of substrates for hybridization including a first linear actuator to position a laser assembly, wherein the laser assembly includes a laser and a galvanometer scan assembly, wherein the galvanometer scan assembly is mounted on a second linear actuator, and a third linear actuator to position a slide holder.

Additional teachings of the invention will be set forth in part in the description which follows, and in part will be obvious from the description, or may be learned by practice of the invention. The teachings of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate various embodiments of the invention and together with the description, serve to explain the principles of the invention.

### BRIEF DESCRIPTION OF THE DRAWINGS

FIG. 1A illustrates a perspective view of an apparatus for preparing a hybridization substrate according to various embodiments;

FIG. 1B illustrates a perspective view of a slide holder according to various embodiments;

FIG. 1C illustrates a cross-sectional view of an apparatus for preparing a hybridization substrate according to various embodiments;

FIG. 1D illustrates a top view of a laser assembly according to various embodiments;

FIG. 1E illustrates a perspective view of portions of a galvanometric assembly according to various embodiments;

FIGS. 2A-2C illustrate a cross-sectional view of the hybridization substrate with a gasket according to various embodiments; and

FIG. 3 illustrates a perspective view of an automated apparatus for preparing a hybridization substrate include a stripper and spotter according to various embodiments.

It is to be understood that both the foregoing general description and the following detailed description are exemplary and explanatory only, and are intended to provide an explanation of various embodiments of the present teachings.

### DESCRIPTION OF VARIOUS EMBODIMENTS

Reference will now be made in detail to the various embodiments of the invention, examples of which are illustrated in the accompanying drawings. Wherever possible, the same reference numbers will be used throughout the drawings to refer to the same or like parts.

The term “collapsing” grammatical variations thereof as used herein refers to treating a portion of a porous layer coupled to a substrate with laser light. Collapsing can include ablating or rapid non-thermal vaporization of porous layer, etching or surface melting of the porous layer, and engraving or surface vaporization of the porous layer. Collapsing can include cutting the porous layer.

The term “porous layer” as used herein refers to materials that exhibit absorption and adsorption qualities for a variety of fluid capture reagents that can be used in connection with the invention. The porous layer can be constructed of any material that is compatible with depositing the array, hybridization fluid, and assay design. The porous layer can include materials such as NYLON, cellulose, nitrocellulose, gel, polymeric, or other porous membrane known in the art of polymer chemistry. The porous layer can be coupled to the substrate using an adhesive such as an epoxy or other bonding agent known in the art of adhesives. The porous layer can be non-luminescent to provide less background light to the luminescent of the hybridization array.

The term “substrate” as used herein refers to a nonporous or non-absorbent material that can be the foundation for coupling the porous layer. The substrate can include material such as glass, fused silica, silicon, plastic, metal, ceramic, or polymeric. The substrate can be pretreated to facilitate coupling to the porous layer. Examples of substrates with nonporous layers include those in U.S. Publication Nos. 2002/0119559A1, 2002/0086307A1, and 2002/0019481A1. The substrate can have any shape including circular, triangular, rectangular, etc.

The term “hybridization” as used herein refers to the process of forming a duplex between two members of specific binding pair. The specific binding pair is frequently complementary or partially complementary strands of a polynucleotide. It will be understood by those skilled in the art of molecular biology that the term “polynucleotide” as used herein includes analogs of naturally occurring polynucleotides and does not covey any limitation of the length of the polynucleotide. One of the polynucleotide strands may be immobilized on a solid substrate. Polynucleotide strands used for hybridization can be labeled with a detectable marker to as facilitate the detection of duplexes. Examples of detectable markers can include, but are not limited to fluorescent dye, radioisotopes, enzyme, or other markers. According to various embodiments, detection can be provided by a CCD camera that detects the detectable markers. Polynucleotide strands used for hybridization can be non-labeled. Hybridization of non-labeled binding pairs can be detected by surface plasmon resonance (SPR). Hybridization can be used for a variety of purposes, including understanding the structure-activity relationship between different materials, detecting and screening single nucleotide polymorphisms (SNPs), and sequencing an unknown material. The term “specific binding pair” refers to a pair of molecules that bind to one another with a specificity that is detectable above background levels of non-specific molecular interactions. Examples of specific binding pairs can include, but are not limited to antibody-antigen (or hapten) pairs, ligand-receptor pairs, biotin-avidin pairs, polynucleotides with complementary base pairs, nucleic acid binding proteins and cognate nucleic sequences, members of multi-protein complexes, and the like. Each specific binding pair can include two members, or additional compounds can specifically bind to either member of a given specific binding pair. The term “hybridization chamber” refers to the reaction volume and its container wherein the hybridization fluid reacts with the polynucleotides in the liquid phase or bound on porous layer.

The term “array” as described herein refers to polynucleotides bound on a substrate to form a microarray. Microarrays can have densities of 4 binding sites per square millimeter or up to 10⁴ binding sites per square millimeter. Binding sites can be positioned on the porous layer by pin spotting, ink-jetting, photo-lithography, and other methods known in the art of high density deposition.

The term “gasket” as used herein refers to gaskets, rings, or seals constructed of a non-porous material that can be used to substantially inhibit flow of liquids. The gasket can be angular or circular in overall shape and/or cross-section. The gasket can form a seal with the substrate thereby forming a boundary for the hybridization chamber. The gasket can be compressible to better form such seal. Compressible materials are known in the material science arts. According to various embodiments, the hybridization frame and/or the detection frame can be constructed of at least one elastomeric material chosen from Silicone Rubber, FDA approved Silicone Rubber, EPDM Rubber, Neoprame (CR) Rubber, SBR Rubber, Nitrile (NBR) Rubber, Butyl Rubber, Hypalon (CSM) Rubber, Polyurethane (PU) Rubber, Viton Rubber, and polydimethylsiloxane (Slygard™ elastomer by Dow Corning).

The term “laser” as used herein refers to gas lasers, solid state lasers, semiconductor lasers, and other materials used to produce laser light, that can include optically pumped lasers, electrically pumped laser, and lasers with other pumping schemes as known in the art of lasers, and/or can include continuous wave lasers, pulsed lasers, and lasers with other operational modes as known in the art of lasers.

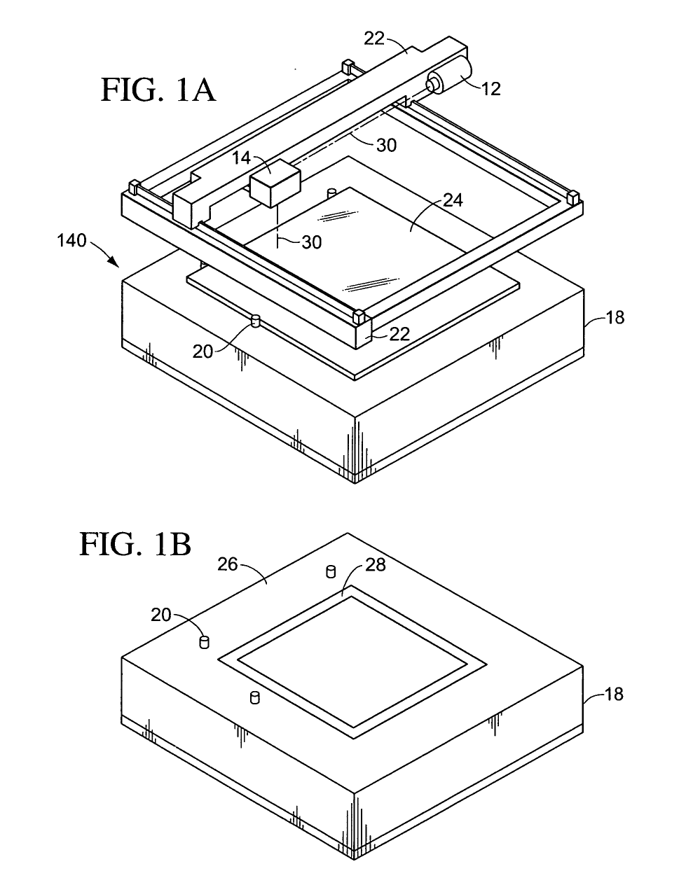

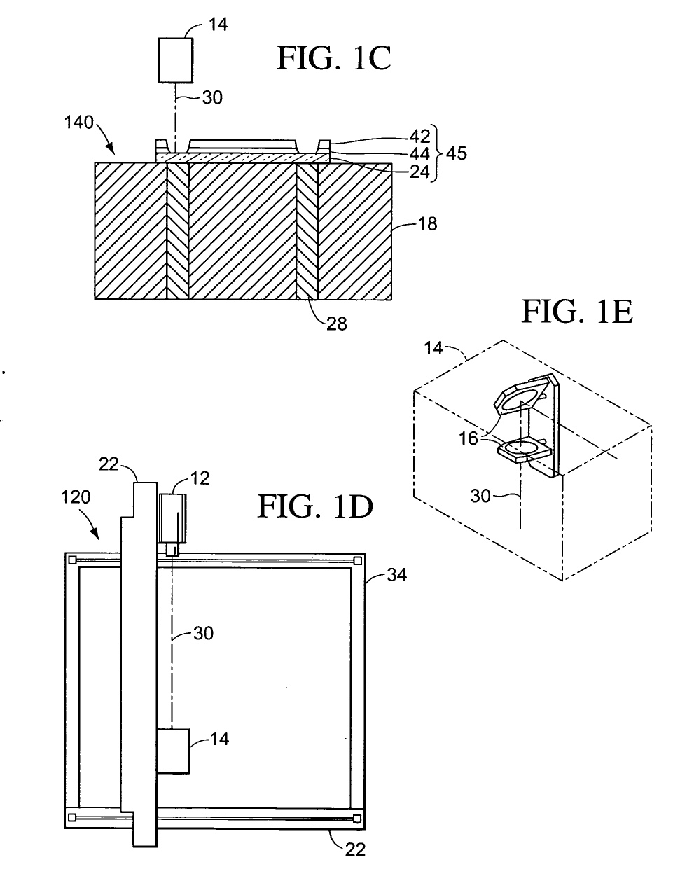

According to various embodiments, as illustrated in FIGS. 1A and 1D, apparatus 100 for preparing a hybridization substrate can include laser assembly 120 and lower portion 140. According to various embodiments, as illustrated in FIG. 1D, laser assembly 120 can include laser 12. According to various embodiments, as illustrated in FIG. 1E, laser assembly 120 can include galvanometer scan assembly 14, including a galvanometer scan head with rotatable mirrors 16. According to various embodiments, laser assembly 120 can include linear actuators 22 and frame 34 to brace the linear actuators 22. According to various embodiments, the galvanometric assembly 14 and/or the linear actuators 22 can position the laser light 30 to collapse a moat 43. According to various embodiments, moat 43 can be in the form of a rectangle (see FIG. 1B), but also can be in the form of a square, circle, triangle, or any form needed to bound the portion of the porous layer where the array is to be deposited. According to various embodiments, the moat can be multiple annular moats. The annular moats can be concentric or adjacent.

According to various embodiments, the laser light can be positioned by moving the laser with linear actuators without the aid of a galvanometer scan assembly. According to various embodiments, the laser light can be positioned with a galvanometer scan assembly without the aid of linear actuators. According to various embodiments, the mirrors can be fixed and not rotatable.

According to various embodiments, laser assembly 120 can electro-mechanical proximity switches, cameras, mechanical threads, and the like can be utilized to provide a means to verify that laser assembly 120 to collapses moat 43 as instructed by the computer controlling the laser assembly 120. Electronics can then process such information and provide feedback to the mechanism driving laser assembly 120 and/or lower portion 140, to modify the collapsing to provide the desired form of the moat 43.

According to various embodiments, a localized vacuum head can be positioned directly over or adjacent to the collapsing site to remove gasses generated by the collapse of the porous layer. According to various embodiments, the vacuum head can be mounted adjacent to the galvanometer scan assembly and be capable to following the laser beam to maintain proximity to the portion of the porous layer being collapsed. According to various embodiments, the vacuum head can reduce contamination of the porous layer surrounding the portion collapsed by products of the collapsing process.

According to various embodiments, lower portion 140 can include slide holder 18 and pegs 20. According to various embodiments, pegs 20 and slide holder 18 can be configured to receive slide 45. According to various embodiments, pegs 20 can be positioned upon slide holder 18 in a manner to orient slide 45 in accordance with the requirements of the particular collapse of porous layer desired.

According to various embodiments, as illustrated in FIG. 1B, slide holder 18 can include at least one conductive portion 26 and at least one non-conductive portion 28. Conductive portion 26 can conduct heat away from various portions of slide holder 18. Accordingly, more heat can be retained at non-conductive portion 28. This retention of heat facilitates the focus of heat on certain portions of slide 45, when placed upon slide holder 18. According to various embodiments, non-conductive portion 28 can comprise a shape roughly corresponding to the shape of moat 43. According to various embodiments, heat generated in collapsing moat 43 by laser light 30 on slide 45 positioned on slide holder 18 can be substantially retained by non-conductive portion 28. According to various embodiments, slide holder 18 can include both conductive and non-conductive portions, conductive portions only, or non-conductive portions only. According to various embodiments, laser light 30 does not substantially generate heat in collapsing moat 43, reducing the need for slide holder 18 to have portions with different conductivity.

According to various embodiments, as illustrated in FIG. 1C, laser assembly 120 can move toward lower portion 140. This movement can be accomplished by coupling upper portion 120 and/or lower portion 140 to a linear actuator (not shown). Operation of the linear actuator is well known to those skilled in the art and includes hydraulic, electric and/or mechanical action. According to various embodiments, the laser light 30 collapses moat 43. According to various embodiments, slide 45 can include substrate 24, adhesive layer 44, and/or porous layer 42. Porous layer 42 can be the foundation upon which an array can be positioned. According to various embodiments, adhesive layer 44 can be coupled substrate 24 to porous layer 42 and can be made from an epoxy.

According to various embodiments, moat 43 can be formed by collapsing a portion of porous layer 42 with laser light 30 from laser 12. According to various embodiments, laser light 30 can collapse porous layer 42 and/or adhesive layer 44 without collapsing substrate 24. According to various embodiments, depending on the width of the beam of laser light 30 collapsing can take several passes of laser light 30 to collapse the entire width of moat 43. According to various embodiments, the collapsing of moat 43 can be provided by raster, vector, or a combination of raster and vector process of laser light 30. According to various embodiments, the raster process can collapse an area wider than the width of the beam of laser light 30 by overlapping adjacent passes. According to various embodiments, the vector process can collapse in a continuous discrete pass. The continuous passes can be overlapped to collapse a area wider than the width of the beam of laser light 30.

According to various embodiments, laser assemblies can include the different laser marking products available on the market, for example, Legend 32EX from Epilog Laser, Inc. (Golden, Colo.), Vectormark from Borries Marking Systems GmbH (Pliezhausen, Germany), GraphiXscan Laser 500 from Viable Systems, Inc. (Medfield, Mass.), and Lasonall Marker from Ostling Technologies, Inc. (Chillicothe, Ohio). According to various embodiments, the laser power, laser head speed, and focus can determine the depth of collapse and can be regulated on the particular laser assembly because power is a function of the percentage of maximum power of the laser and laser head speed. According to various embodiments, the laser can be Nd:YAG or CO₂. According to various embodiments, the laser power can vary from 5 watts to 500 watts.

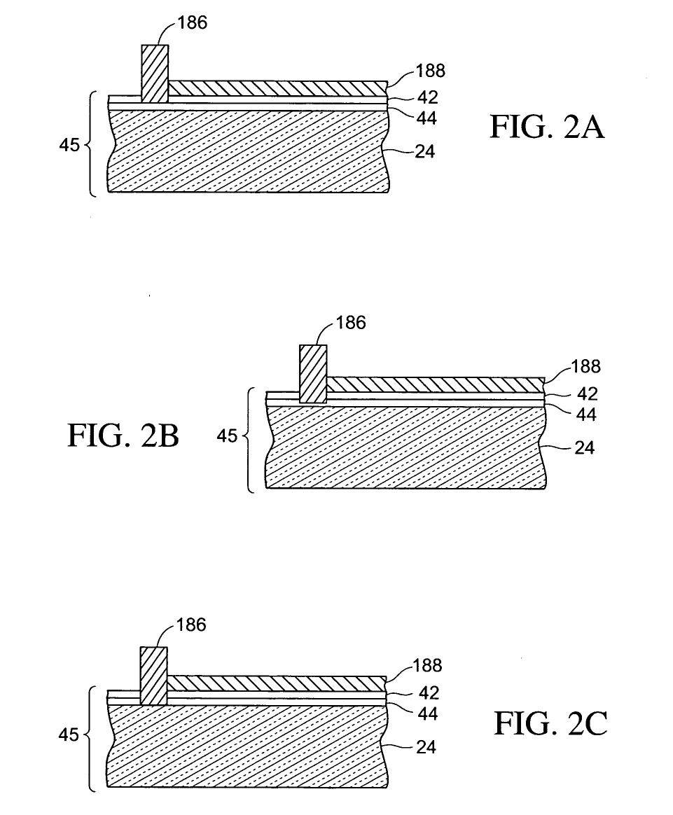

According to various embodiments, moat 43 can extend through the entirety of porous layer 42 and/or adhesive layer 44, or can extend through a portion of each, or both as illustrated in FIGS. 2A-2C. According to various embodiments, the laser assembly can vary the depth of collapse. According to various embodiments, a sensor on the laser assembly can be coupled to determine the depth of collapse by sensing the distance between the laser and the slide 45.

According to various embodiments, a gasket 186 can be situated within moat 43 to act as a barrier to various liquids. According to various embodiments, gasket 186 can be adhered to various layers of slide 45. According to various embodiments, the gasket can be held under pressure against various layers of slide 45.

According to various embodiments, FIGS. 2A-2C illustrate a cross-section of the substrate, porous layer and gasket. FIGS. 2A-2C illustrate that slide 45 can include substrate 24, adhesive layer 44, and porous layer 42. The array 188 binds to nonporous layer 42. Porous layer 42 can sprayed on, laminated on, deposited on via chemical vapor deposition, or deposited on via electrostatic deposition on the substrate 24 and adhesive layer 44. The adhesive layer 44 can be hydrophobic to seal the hybridization chamber, such as a pressure sensitive acrylic adhesive. Gasket 186 can be adhered to the slide 45 via its own adhesive or by using the adhesive properties of adhesive layer and/or porous layer. According to various embodiments, FIG. 2A illustrates gasket 186 adhered to the interface of porous layer 42 and adhesive layer 44. According to various embodiments, FIG. 2B illustrates gasket 186 adhered to adhesive layer 44. According to various embodiments, FIG. 2C illustrates gasket 186 adhered to the interface of adhesive layer 44 and substrate 24. According to various embodiments, each of these adhesion contacts provides a seal for a boundary of the hybridization chamber.

According to various embodiments, apparatus 100 can be part of an automated array printer. According to various embodiments, apparatus 100 can be incorporated into a robotic platform designed to prepare hybridization substrate and position arrays in an automated fashion.

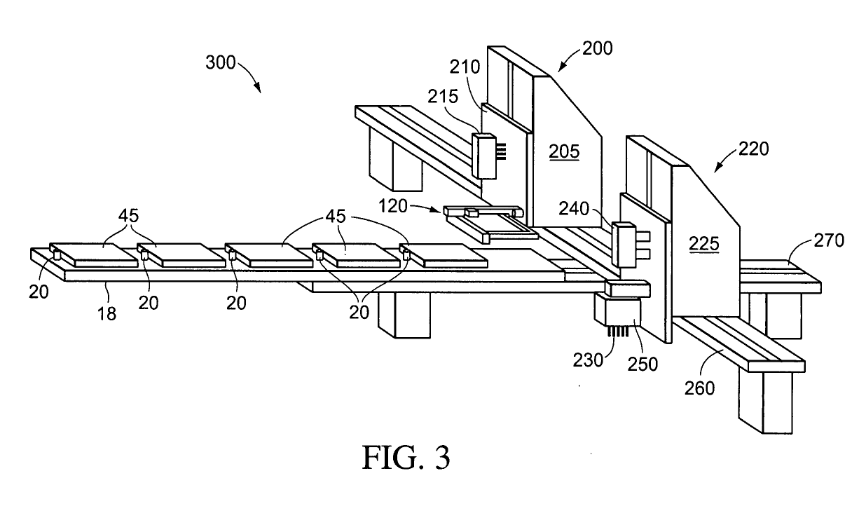

According to various embodiments, as illustrated in FIG. 3, the collapsing process can be automated by system 300 for several slides 45 on slide holder 18 mounted on a linear actuator 270. Each slide can include alignment pegs 20. Collapsing assembly 200 can include linear actuator 205 with mounting plate 210. Laser assembly 140 can be mounted on mounting plate 210. The linear actuator 260 can position collapsing assembly 200 and linear actuator 270 can position slide holder 18 to align laser assembly 140 with slide 45. The linear actuator 205 can approach slide holder 18 until laser assembly 140 reaches the distance to slide 45 sufficient to collapse the moats. Laser assembly 140 can include a sensor to provide an electrical signal to stop linear actuator 205 so that when the sufficient distance is met. Collapsing assembly 200 can include a camera 215 to control the linear actuator and/or provide image information related to the collapsing. According to various embodiments, system 300 can include an array spotter 220 with a linear actuator 225 to position spotting head 250 so that tips 230 can deposit the array on the porous layer of slide 45. Spotter 220 can include camera 240 to provide image information related to proximity of spotting the array relative to the moat.

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

It is noted that, as used in this specification and the appended claims, the singular forms “a,” “an,” and “the,” include plural referents unless expressly and unequivocally limited to one referent. Thus, for example, reference to “a monomer” includes two or more monomers.

Other various embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method for preparing a substrate for hybridization, the method comprising:
positioning a porous layer on the substrate; and
collapsing a moat in the porous layer with a laser,
wherein the moat is adapted to bound a portion of the porous layer on which an array can be positioned.

2. The method of claim 1, wherein the collapsing occurs without hearing the porous layer.

3. The method of claim 1, wherein the porous layer comprises nylon.

4. A method for manufacturing, comprising:
providing a substrate comprising a porous layer, wherein the porous layer is adapted for depositing an array;
providing a laser assembly, wherein the laser assembly comprises laser; and
collapsing the moat in the porous layer with the laser.

5. The method of claim 4, wherein laser assembly further comprises at least one of a linear actuator and a galvanometer scan assembly.

6. A method for preparing a hybridization chamber, comprising:
providing a substrate comprising a porous layer with a moat collapsed with a laser;
positioning an array on a portion of the porous layer bound by the moat; and
positioning a gasket in the moat to provide a nonporous seal.

7. An apparatus for preparing a hybridization substrate, comprising:
a laser assembly adapted to collapse a moat in a porous layer on the substrate; and
a galvanometer scan assembly adapted to position laser light from the laser assembly on the porous layer.

8. The apparatus of claim 7, further comprising a thermal path on a bottom portion of the apparatus.

9. The apparatus of claim 7, further comprising a linear actuator.

10. The apparatus of claim 7, further comprising a vacuum head.

11. The apparatus of claim 10, wherein the vacuum head is positioned adjacent to the galvanometer scan assembly.

12. A laser assembly, comprising:
a laser adapted to collapse a moat in a porous layer of a hybridization substrate;
a mechanism to position the laser light on a portion of the porous layer.

13. The laser assembly of claim 12, wherein the mechanism comprises at least one of a linear actuator and a galvanometer scan assembly.

14. A substrate for hybridization, comprising:
a porous layer, wherein the porous layer is adapted for depositing an array; and
a moat in the porous layer, wherein the moat is collapsed by laser.

15. The substrate of claim 14, further comprising an array.

16. The substrate of claim 15, further comprising a hybridization fluid.

17. An apparatus for preparing a substrate for hybridization comprising means for providing a moat in a porous layer on a substrate by laser means, wherein the porous layer is adapted for depositing an array.

18. The apparatus of claim 17, wherein the means for providing a moat comprise means for positioning the laser means.

19. The apparatus of claim 17, further comprising means for spotting the array on the substrate.

20. A system for automated preparation of substrates for hybridization comprising:
a first linear actuator to position a laser assembly, wherein the laser assembly comprises a laser and a galvanometer scan assembly, wherein the galvanometer scan assembly is mounted on a second linear actuator, and
a third linear actuator to position a slide holder.

21. The system of claim 20, further comprising a fourth linear actuator to position a spotting head.

22. The system of claim 20, further comprising a camera to inspect a moat on the substrate.
